(19) United States
(12) Patent Application Publication
(10) Pub. No.: US 2023/0348835 A1
(43) Pub. Date: Nov. 2, 2023

(54) Method of Culturing Alga and Alga Culture System

(71) Applicant: National University Corporation Hokkaido University, Hokkaido (JP)

(72) Inventors: Masahiro Sato, Sapporo-shi, Hokkaido (JP); Takumi Nakashima, Sapporo-shi, Hokkaido (JP); Kazuei Ishii, Sapporo-shi, Hokkaido (JP); Satoru Ochiai, Sapporo-shi, Hokkaido (JP)

(73) Assignee: National University Corporation Hokkaido University, Hokkaido (JP)

(21) Appl. No.: 18/025,769

(22) PCT Filed: Sep. 10, 2021
(86) PCT No.: PCT/JP2021/033429

§ 371 Date: Mar. 10, 2023

(30) Foreign Application Priority Data
Sep. 11, 2020 (JP) 2020-153276
Oct. 9, 2021 (JP) JP2021/033429

(51) Int. Cl. C12N 1/12; B01D 69/02; B01D 61/14; C12M 1/00; C12M 1/34; B01D 61/24

(52) U.S. Cl. CPC C12M 29/04; B01D 61/147; B01D 61/243; B01D 69/02; C12M 41/32; C12N 1/12; B01D 2325/0283

(57) ABSTRACT

The present invention provides a method of culturing an alga that enables practical realization of low-cost and space-saving production of biofuels and bioenergy by culturing the alga using a reaction tank equipped with a digestive solution tank comprising digestive solution containing high concentration of nutrient salts, a membrane with a pore size of 0.45 µm or less and a culture tank comprising culture solution and the alga, which comprises consuming the nutrient salts by the alga in the culture tank to maintain the difference in concentration the nutrient salts between the digestive solution tank and the culture tank and supplying the nutrient salts contained in the digestive solution through the membrane into the culture solution by the diffusion depending on the concentration difference as well as an alga culture system therefor.

### TECHNICAL FIELD

The present invention relates to a method of culturing an alga and an alga culture system.

### BACKGROUND ART

Various studies for culturing an alga such as a microalga (e.g., an indigenous microalga) and producing biofuels and bioenergy from the cultured alga used as a raw material have been performed. In order to produce biofuels and bioenergy from an alga on a practical and commercial scale, it is necessary to culture and extract a large amount of an alga. However, the costs for culturing and extracting a large amount of an alga are high. Among the costs, it is thought that one of main causes of the high costs is the cost for securing nutrient salts required for culturing an alga. Hence, the obtained nutrient salts, for example, the nutrient salts in livestock manure and the fermentation residue after the methane fermentation of livestock manure have often been used in not the supply to the culture of an alga but agricultural application because of the emphasis on economic rationality.

As methods of extracting nutrient salts from wastewater containing high concentration of nutrient salts, hydroxyapatite (HAP) method, magnesium ammonium phosphate (MAP) method and ammonia stripping method have been known (Non-Patent Documents 1 to 3). The HAP and MAP methods are methods of extracting phosphorus from sewage sludge or night soil as wastewater containing high concentration of nutrient salts. The methods can be used to extract phosphorus and nitrogen components as precipitates (solids). On the other hand, the methods have often been used in the process of treating wastewater, which requires pre-treatment processes such as the removal of turbidity components in the wastewater. The ammonia stripping method is a method of transferring high concentration of ammonia nitrogen contained in the wastewater as ammonia gas from liquid phase to gas phase to collect ammonia in the gas. Also, the method has often been used in the process of treating wastewater and the collected ammonia is treated by catalytic degradation.

Some attempts to develop various methods of extracting nutrient salts from wastewater through a membrane have recently been done. In the methods, MF membrane (micro filtration membrane), UF membrane (ultrafiltration membrane), NF membrane (nano filtration membrane) are used as a membrane, and the nutrient salts can move by differential pressure. The methods treated through a membrane can be used in the limited space and at relatively low cost, and thus have been used in the treatment of livestock manure and fermentation residue thereof (Non-Patent Document 4). However, there was the problem that the static pressure produced by the difference in hydraulic head caused clogging (fouling) on the membrane surface and delayed the movement of nutrient salts. As a result, the methods could not be used continuously.

In addition, a method of extracting nutrient salts in combination with the processes of membrane separation, electrodialysis and distillation has been known as a method of using a membrane (Non-Patent Document 5). However, the method was required to be performed through some processes, and thus there was the problem that the cost for performing the method was high. As a method of extracting nutrient salts efficiently from wastewater with a low possibility of clogging, the method of using FO membrane (Forward Osmosis membrane) which is the reverse application of the principle of RO membrane (Reverse Osmosis membrane) used for salt water desalination has been known. On the other hand, it was necessary to set the concentration of nutrient salts on the extracted side to a high level (sodium hydroxide: 3.5 M) in order to extract the nutrient salts by the use of osmotic pressure, and thus the method had the problem that the use of the extracted nitrogen components was limited.

Conventional methods of extracting nutrient salts had various problems. Hence, the methods could not be sufficiently used for culturing and extracting an alga. As a result, it has been strongly desired to develop an effective method of providing nutrient salts at low costs. A method of culturing an alga by supplying nutrient salts extracted from digestive solution through a membrane with a pore size of 0.45 µm or less to culture solution by the diffusion driven by the difference in concentration of nutrient salts between the digestive solution and the culture solution has not been reported as a method of using a membrane.

### PRIOR ART DOCUMENTS

### Non-Patent Documents

Non-Patent Document 1: Takao HAGINO, Tsuyoshi HIRASHIMA: Development of a Process for Recovering Phosphorus from Sewage Sludge, Resources Processing, 52: 172-182, 2005
Non-Patent Document 2: Hirokazu SHIRAGE: Recovery of Phosphate Using MAP Method, Journal of Environmental Biotechnology, Vol. 4, No. 2, 109-115, 2005
Non-Patent Document 3: Junichi TAKAHASHI: Conversion of unused resources to animal feed by ammonia stripping of fermented and digested liquid from biogas plant fermentation, Journal of agricultural and food technology, Vol.3, No.33. 5-10, 2007
Non-Patent Document 4: Mehta, C. M., Khunjar, W. O., Nguyen, V., Tait, S., & Batstone, D. J. (2015, February 16). Technologies to recover nutrients from waste streams: A critical review. Critical Reviews in Environmental Science and Technology, Vol. 45, pp. 385-427
Non-Patent Document 5: Mitsuyasu YABE: Separated and concentrated collection of fertilizer components from methane fermentation digestive solution, Agricultural biotechnology, Vol. 3, No. 4, 370-374, 2019

### SUMMARY OF INVENTION

### Problem to Be Solved by the Invention

An object of the present invention is to provide a method of culturing a large amount of an alga at low cost that enables practical realization of low-cost and space-saving production of biofuels and bioenergy by culturing the alga continuously, which comprises supplying nutrient salts from digestive solution containing high concentration of nutrient salts to culture solution at a supply rate suitable for the culture of alga as well as an alga culture system therefor, besides a new method of supplying and extracting nutrient salts through a membrane.

### Means for Solving the Problems

The present inventors have extensively studied to reach the above object, and then have found that the setting of a membrane with a pore size of 0.45 µm between a digestive solution tank and a culture tank in a reaction tank can prevent the clogging on the membrane and keep the volumes of each solution in the digestive solution tank and the culture tank at the same level, and thus an alga can be cultured continuously by repeating the cycle in which high concentration of nutrient salts in digestive solution is supplied from the digestive solution tank through the membrane to the culture tank by the diffusion driven by the difference in concentration of the nutrient salts between the digestive solution tank and the culture tank and the alga consumes the supplied nutrient salts to culture the alga. Based upon the new findings, the present invention has been completed.

That is, the present invention provides the following embodiments.

[Item 1] A method of culturing an alga using a reaction tank equipped with a digestive solution tank comprising digestive solution containing high concentration of nutrient salts, a membrane with a pore size of 0.45 µm or less and a culture tank comprising culture solution and an alga, which comprises supplying the nutrient salts contained in the digestive solution through the membrane into the culture solution by the diffusion driven by the difference in concentration of the nutrient salts between the digestive solution tank and the culture tank.

[Item 2] The method according to the item 1, wherein the supply rate of the nutrient salts is 177 to 188 g-N/m²/d.

[Item 3] The method according to the item 1 or 2, wherein the membrane has an area of 0.0193 m² or more.

[Item 4] The method according to any one of the items 1 to 3, wherein the culture rate of the alga is 49 to 234 g/m³/d.

Item 5] The method according to any one of the items 1 to 4, which further comprises circulating each solution in the digestive solution tank and the culture tank using each pump further equipped in each tank and keeping the volumes of the digestive solution and the culture solution at the same level.

[Item 6] The method according to any one of the items 1 to 5, which further comprises supplying CO₂ into the culture tank.

[Item 7] The method according to any one of the items 1 to 6, which further comprises supplying phosphate ion (PO₄³⁻) into the culture tank.

[Item 8] The method according to any one of the items 1 to 7, wherein the digestive solution is methane fermentation digestive solution.

[Item 9] The method according to any one of the items 1 to 8, wherein the culture solution is tap water without chlorine, groundwater, or river or lake water.

[Item 10] The method according to any one of the items 1 to 9, wherein the alga is a microalga.

[Item 11] The method according to any one of the items 1 to 10, wherein the membrane is micro filtration membrane (MF membrane).

[Item 12] The method according to any one of the items 1 to 11, wherein the nutrient salts comprises one or more salts consisting of ammonia nitrogen, nitrate nitrogen, phosphate phosphorus, orthosilicic acid, potassium, calcium, magnesium and sulfur.

[Item 13] An alga culture system comprising a digestive solution tank, a membrane with a pore size of 0.45 µm or less and a culture tank, wherein the digestive solution tank comprises digestive solution containing high concentration of nutrient salts, the culture tank comprises culture solution and an alga, and the membrane is set as a partition between the digestive solution tank and the culture tank.

[Item 14] The alga culture system according to the item 13, wherein the system maintains the difference in concentration of nutrient salts between the digestive solution tank and the culture tank produced by the consumption of the nutrient salts by the alga in the culture tank and supplies the nutrient salts contained in digestive solution into culture solution by the diffusion driven by the difference in concentration of nutrient salts.

[Item 15] The alga culture system according to the item 13 or 14 which is arranged in the order of the digestive liquid tank, the membrane and the culture tank in a horizontal direction.

[Item 16] The alga culture system according to the item 13 or 14 which is arranged in the order of the digestive liquid tank, the membrane and the culture tank in a vertical direction.

[Item 17] A method of supplying nutrient salts using a reaction tank equipped with a digestive solution tank comprising digestive solution containing high concentration of nutrient salts, a membrane with a pore size of 0.45 µm or less and a culture tank comprising culture solution and an alga, wherein comprises maintaining the difference in concentration of nutrient salts between the digestive solution tank and the culture tank produced by the consumption of the nutrient salts by the alga in the culture tank and supplying the nutrient salts contained in digestive solution through the membrane into culture solution by the diffusion driven by the difference in concentration of nutrient salts

### Effects of the Invention

The present invention can supply the nutrient salts from digestive solution to culture solution at the supply rate and in the required amount suitable for the culture of an alga. Also, the present invention can supply the nutrient salts without the pre-treatment processes such as the removal of turbidity components, and thus can achieve the culture and extraction of an alga at low cost.

In addition, the present invention can culture a large amount of an alga, and thus it is expected to enable the practical realization of the production of biofuels and bioenergy on a commercial scale.

### BRIEF DESCRIPTION OF THE DRAWINGS

FIG. 1 shows a schematic diagram representing an example of an alga culture system of the present invention. FIG. 1(a) is horizontal, and FIG. 1(b) is vertical. Q_{d} represents the amount of water flowed into digestive solution tank, and Q_{c} represents the amount of water flowed into culture tank.

FIG. 2 shows the changes over time in fluorescence intensity for each culture solution prepared from 20-fold diluted digestive solution, 50-fold diluted digestive solution, 100-fold diluted digestive solution and CSi medium.

FIG. 3 shows a diagram of an experimental apparatus for culturing an indigenous microalga by the addition of mixed gas (CO₂ gas) or air. The experimental apparatus for culturing the microalga by the addition of mixed gas (CO₂ gas) is a vial with butyl rubber aluminum seal stopper (Volume: 228 mL) comprising dilute digestive solution (100 mL) and microalga solution (20 mL) with an aluminum gas bag (400 mL) containing a mixture of CO₂ gas and air with a CO₂ gas concentration of about 10% connected by a tube and tube fitting, and the experimental apparatus for culturing the microalga by the addition of air is a vial with breathable silicone stopper comprising dilute digestive solution (100 mL) and microalga solution (20 mL).

FIG. 4 shows a diagram of the experimental apparatus for extracting turbidity components and nutrient salts in digestive solution. H represents the volume of solution (level of solution), and Φ40 represents a diameter of 40 mm.

FIG. 5 shows the changes over time in the light transmittance (%) for the culture solution obtained using the experimental apparatus shown in FIG. 4. represents the changes over time in the light transmittance for the culture solution in the first experiment, and • represents the changes over time in the light transmittance for the culture solution in the second experiment. The line on a transmittance of 34.9% means the light transmittance for 50-fold diluted digestive solution optimal for the culture of a microalga as shown in Example 1.

FIG. 6 shows the changes over time in the ammonium ion (NH₄+) amounts (g) in the digestive solution tank and the culture tank. ◊ represents the changes over time in the ammonium ion (NH₄+) amounts (g) in the digestive solution tank in the first experiment, ◆ represents the changes over time in the ammonium ion (NH₄+) amounts (g) in the digestive solution tank in the second experiment, Δ represents the changes over time in the ammonium ion (NH₄+) amounts (g) in the culture tank in the first experiment, and ▲ represents the changes over time in the ammonium ion (NH₄+) amounts (g) in the culture tank in the second experiment.

FIG. 7 shows the changes over time in the potassium ion (K+) amounts (g) in the digestive solution tank and the culture tank. ◊ represents the changes over time in the potassium ion (K⁺) amounts (g) in the digestive solution tank in the first experiment, ◆ represents the changes over time in the potassium ion (K⁺) amounts (g) in the digestive solution tank in the second experiment, Δ represents the changes over time in the potassium ion (K⁺) amounts (g) in the culture tank in the first experiment, and ▲ represents the changes over time in the potassium ion (K+) amounts (g) in the culture tank in the second experiment.

FIG. 8 shows the amount of nutrient salts moved from the digestive solution tank to the culture tank per unit time and unit area (separation flux) and the amount of NH₄⁺ associated with the movement of water from the culture tank to the digestive solution tank (movement flux).

FIG. 9 shows the amounts of microalga in the culture tank from Day 1 to Day 28. The amounts of microalga on Day 8 to Day 10 are not measured. In the culture period, represents the day when distilled water was added, Δ represents the day when the digestive solution was replaced, and □ represents the day when the nutrient salts were added.

FIG. 10 shows the PO₄³⁻ amounts in the digestive solution and the culture solution from Day 1 to Day 28. The PO₄³⁻ amounts on Day 8 and Day 9 are not measured. In the graph, ■ represents the PO₄³⁻ amounts in the digestive solution, and □ represents the PO₄³⁻ amounts in the culture solution. In the culture period, ○represents the day when distilled water was added, Δ represents the day when the digestive solution was replaced, and □ represents the day when the nutrient salts were added.

FIG. 11 shows the NH₄⁺ amounts in the digestive solution and the culture solution on Day 1 to Day 28. The NH₄⁺ amounts on Day 8 and Day 9 are not measured. In the graph, ■ represents the NH₄+ amounts in the digestive solution, and □ represents the NH₄+ amounts in the culture solution. In the culture period, ○ represents the day when distilled water was added, Δ represents the day when the digestive solution was replaced, and □ represents the day when the nutrient salts were added.

### DESCRIPTION OF EMBODIMENTS

Hereinafter, the present invention is specifically explained.

The present invention provides a method of culturing an alga using a reaction tank equipped with a digestive solution tank comprising digestive solution containing high concentration of nutrient salts, a membrane with a pore size of 0.45 µm or less, and a culture solution tank comprising culture solution and an alga.

The method of culturing an alga of the present invention comprises supplying nutrient salts contained in digestive solution through a membrane into culture solution by the diffusion driven by the difference in concentration of the nutrient salts between the digestive solution tank and the culture tank produced by the consumption of the nutrient salts by an alga in the culture tank.

As used herein, the term “alga” refers to an organism(s) that produces oxygen through photosynthesis, which mainly excludes terrestrial plants such as mosses, ferns and seed plants. The alga of the present invention may be a microorganism(s) performing biosynthesis such as euglena. The alga is not particularly limited and may be appropriately selected for the purpose.

The alga of the present invention is preferably a microalga (e.g., an indigenous microalga). The microalga as used herein refers to a microscopic alga invisible to the human eye. The microalga may be prokaryotic or eukaryotic.

Examples of the microalga include a microalga belonging to any groups such as Chlorophyta, Glaucophyta, Rhodophyta, Chlorarachniophyta, Euglenophyta, Cryptophyta, Phaeophyta, Haptophyta, Heterokontophyta, Dinophyta, Chromerida and Cyanobacteria. The group to which the microalga belongs may be undetermined as long as it is found that the microalga belongs to any of the groups or is closely related from the molecular phylogenetic analysis.

In the method of the present invention, one type of alga may be used alone, or two or more types of algae may be used in combination. When the alga is in a symbiotic relationship with another organism, it may be used with the organism.

The method of obtaining a microalga is not particularly limited and may be appropriately selected for the purpose. Examples thereof include a method of extracting a microalga from the natural world, a method of using commercially available products, and a method of obtaining a microalga from the culture extraction or the depositary institution.

The alga cultured in the method of culturing an alga of the present invention may be collected by any commonly-used method such as centrifugal separation from culture solution, sedimentation with a flocculant and membrane separation. Also, the alga may be collected by depositing biofilms formed on the surface of culture solution.

As used herein, the term “digestive solution” refers to a residue obtained after the fermentation of a staring a raw material such as livestock waste, food processing residue, used cooking oil, kitchen waste, sewage sludge, night soil and sludge in a septic tank in a biogas plant (BGP). For example, the digestive solution is methane fermentation digestive solution. Also, the digestive solution of the present invention is easy to secure raw materials in large amounts. It is preferably digestive solution derived from livestock waste (e.g., cattle manure).

As used herein, the term “nutrient salts” refers to salts required as the nutrients for an alga (e.g., a microalga). Examples of the nutrient salts include nitrogen such as ammonia nitrogen, nitrate nitrogen, nitrite nitrogen and organic nitrogen, phosphorus such as phosphate phosphorus and organic phosphorus, silicon such as orthosilicic acid, potassium, calcium, magnesium and sulfur. In the present invention, the nutrient salts may be used as a nutrient source for the growth of an alga.

As used herein, the term “culture solution” refers to solution with high light transmittance. Examples of the culture solution include tap water without chlorine, groundwater and river or lake water. The light transmittance for the culture solution of the present invention is preferably 34.9% or more. For example, the culture solution is prepared by using distilled water and inoculating water collected from the bottom layer of a pond located on the campus of HOKKAIDO UNIVERSITY as the initial indigenous microalga.

The culture solution of the present invention maintains nutrient salts at low concentration, for example, by repeating the cycle in which nutrient salts are supplied from the digestive solution tank into the culture tank and an indigenous microalga consumes the supplied nutrient salts to culture the microalga.

As used herein, the term “membrane (filter)” refers to a partition between the digestive solution tank and the culture tank. Examples of the membrane of the present invention include micro filtration membrane (MF membrane), ultrafiltration membrane (UF membrane) and nano filtration membrane (NF membrane). The membrane of the present invention is preferably a membrane with a pore size of 0.45 µm or less. When the pore size of a membrane exceeds 0.45 µm, the turbidity component in digestive solution is moved to culture solution. As a result, the light transmittance for the culture solution is decreased and the biosynthesis of an alga is inhibited. In addition, the membrane of the present invention has an area of 0.0193 m² or more per a tank volume of 1 m³ and preferably an area of 0.0256 m² or more.

As used herein, the term “turbidity component” refers to a material for providing turbidity into a solution with a particle size of greater than 0.45 µm. Examples of the turbidity component of the present invention include particulate organic material, plankton and other microorganism and suspended material.

The method of culturing an alga of the present invention can supply nutrient salts to the culture solution at a supply rate of 177 to 188 g-N/m²/d.

The method of culturing an alga of the present invention can culture the alga at a rate (growth rate) of 49 to 234 g/m³/d. In the present invention, the culture rate of alga may be 49 to 73 g/m³/d, 49 to 78 g/m³/d, 49 to 93 g/m³/d, 49 to 126 g/m³/d, 73 to 93 g/m³/d, 73 to 126 g/m³/d, 73 to 234 g/m³/d, 78 to 93 g/m³/d, 78 to 126 g/m³/d, 78 to 234 g/m³/d, 93 to 126 g/m³/d, 93 to 234 g/m³/d or 126 to 234 g/m³/d.

The method of culturing an alga of the present invention can increase the culture rate of alga by supplying phosphorus source, preferably phosphate ion (PO₄³⁻) at an appropriate rate into the culture tank depending on the amount of nitrogen supplied into the digestive solution. For example, the ratio of amount of nitrogen supplied into the digestive solution : amount of phosphate ion supplied into the culture tank is 7:1.

The method of culturing an of the present invention can circulate digestive solution and culture solution in the digestive solution tank and the culture tank, respectively, using a stirring device, preferably a pump, keep the volumes of digestive solution and culture solution at the same level, and maintain the difference in the concentration of nutrient salts between the digestive solution tank and the culture tank.

The method of culturing an alga of the present invention can enhance the culture of alga by supplying carbon source, preferably CO₂ into the culture tank.

The method of culturing an alga of the present invention can enhance the culture of alga by supplying phosphorus source, preferably phosphate ion (PO₄³⁻) at an appropriate rate into the culture tank depending on the amount of nitrogen supplied into the digestive solution. For example, the ratio of amount of nitrogen supplied into the digestive solution : amount of phosphate ion supplied into the culture tank is 7:1. For example, in the method of culturing an alga of the present invention, the culture rate of alga is increased by supplying phosphate ion at a concentration of 1.05 mol/m³ into the culture tank to enhance the culture of alga.

As used herein, the “alga culture system” is equipped with a digestive solution tank, a membrane (filter) and a culture tank. The digestive solution tank comprises digestive solution containing high concentration of salts, and the culture tank comprises culture solution and an alga. The digestive solution tank and culture tank may have one or more devices such as a stirring device, a device for controlling temperature, a device for adjusting pH, a device for measuring turbidity, a device for controlling light and a device for measuring the concentration of specific gas such as CO₂. The membrane is set between the digestive solution tank and the culture tank, and the pore size thereof is preferably 0.45 µm or less.

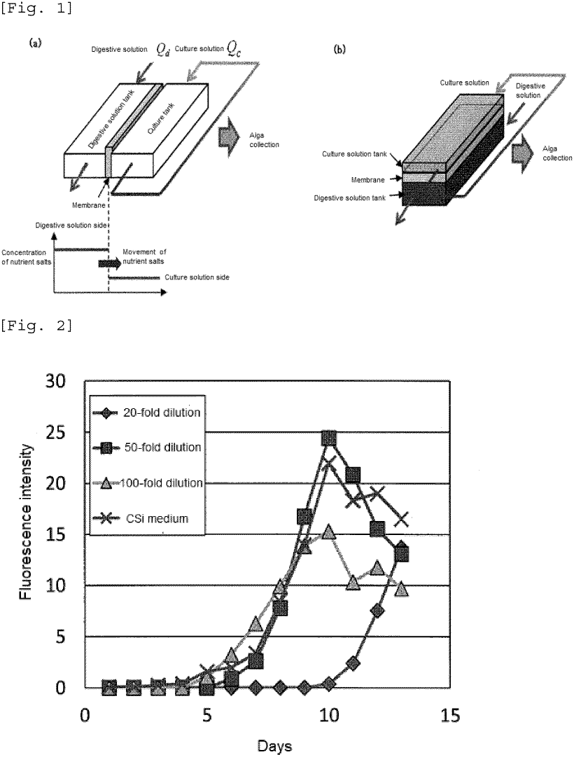

The alga culture system as used herein may be arranged in the order of the digestive solution tank, the filter and the culture tank in the horizontal direction or may be arranged in their order in the vertical direction, as shown in FIGS. 1 (a) and (b).

The alga culture system as used herein can be used, for example, by adding 600 mL each of digestive solution and distilled water into digestive solution tank and culture tank separated by a filter in a clear pipe with a diameter of 40 mm made from polyvinyl chloride with a flange with a packing and the 0.45 µm filter between the separated flanges, respectively, keeping the volumes of solution in both tanks at the same level, and circulating each solution from the bottom to the top of each tank at 400 mL/min by each pump for stirring the inside of each tank.

The alga culture system of the present invention can maintain the difference in concentration of the nutrient salts between the digestive solution tank and the culture tank as the alga in the culture tank consumes the nutrient salts, and thus can supply the nutrient salts contained in digestive solution into culture solution by the diffusion driven by the difference in concentration of nutrient salts.

The alga culture system of the present invention can supply the nutrient salts into the culture tank at a supply rate suitable for the culture of alga, resulting in low-cost culture.

The alga culture system of the present invention can minimize the movement of turbidity components even when digestive solution containing high concentration of nutrient salts comprising a large amount of turbidity components is used.

In the alga culture system of the present invention shown in FIG. 1, the change in concentration of the nutrient salts in the digestive solution tank can be calculated according to Formula (1):

$${V}_{d} \frac{d {C}_{s d}}{d t} = {Q}_{d} \left({C}_{s d}^{i n} - {C}_{s d}\right) - F {A}_{f} \qquad \text{(1)}$$

In the alga culture system of the present invention shown in FIG. 1, the change in concentration of the nutrient salts in the culture tank can be calculated according to Formula (2):

$${V}_{c} \frac{d {C}_{s c}}{d t} = F {A}_{f} - {r}_{x} {Y}_{x s} {V}_{c} \qquad \text{(2)}$$

In the alga culture system of the present invention shown in FIG. 1, the change in concentration of the alga in the culture tank can be calculated according to Formula (3):

$${V}_{c} \frac{d {C}_{x}}{d t} = {r}_{x} {V}_{c} - {C}_{x} {Q}_{c} \qquad \text{(3)}$$

In the alga culture system of the present invention shown in FIG. 1, the separation flux of nutrient salts (the amount of nutrient salts moved from the digestive solution tank to the culture tank per unit time and unit area) can be calculated according to Formula (4):

$$F = k \left({c}_{s d} - {C}_{s c}\right) \qquad \text{(4)}$$

In the above formulae, V represents the volume of a tank (V_{d} represents the volume of digestive solution tank, V_{c} represents the volume of culture tank), Cₛ represents the concentration of nutrient salts (C_{s d} represents the concentration of nutrient salts in digestive solution tank, C_{cd} represents the concentration of nutrient salts in culture tank), Cₓ represents the concentration of an alga, Q represents the amount of water flowed into tank (Q_{d} represents the amount of water flowed into digestive solution tank, Q_{c} represents the amount of water flowed into culture tank), rₓ represents the growth rate of microalga, Yₓₛ represents the amount of consumed nutrient salts per microalga, F represents separation flux of nutrient salts, A_{f} represents filter area, and k represents movement rate coefficient of membrane.

When the growth rate of alga and the consumed nutrient salts per alga in each assumed concentration at the steady state are constant, the concentration of nutrient salts into the digestive solution tank can be calculated according to Formula (5):

$${C}_{s d} = {C}_{s d}^{i n} - \frac{{r}_{x} {Y}_{x s} {V}_{c}}{{Q}_{d}} \qquad \text{(5)}$$

When the growth rate of alga and the consumed nutrient salts per alga in each assumed concentration at the steady state are constant, the concentration of nutrient salts into the culture tank can be calculated according to Formula (6):

$${C}_{s c} = {C}_{s d}^{i n} - \frac{{r}_{x} {Y}_{x s} {V}_{c}}{{Q}_{d}} - \frac{{r}_{x} {Y}_{x s} {V}_{c}}{k {A}_{f}} \qquad \text{(6)}$$

When the growth rate of alga and the consumed nutrient salts per alga in each assumed concentration at the steady state are constant, the concentration of alga can be calculated according to Formula (7):

$${C}_{x} = \frac{{r}_{x} {V}_{c}}{{Q}_{c}} \qquad \text{(7)}$$

The present invention provides a method of supplying nutrient salts using a reaction tank equipped with a digestive solution tank comprising digestive solution containing high concentration of nutrient salts, a membrane with a pore size of 0.45 µm or less and a culture tank comprising culture solution and an alga, which comprises maintaining the difference in concentration of nutrient salts between the digestive solution tank and the culture tank produced by the consumption of the nutrient salts by the alga in the culture tank and supplying the nutrient salts contained in digestive solution through the membrane into culture solution by the diffusion driven by the difference in concentration of nutrient salts.

### EXAMPLES

Hereinafter, the present invention is specifically explained by Examples to better understand the invention, but is not limited thereto.

### Example 1: Study of Digestive Solution Used for Culturing Alga

To 50 mL of a culture (methane fermentation digestive solution from cattle manure and standard medium (CSi)) was added 10 mL of environmental water (collected from the bottom layer of a pond located on the campus of HOKKAIDO UNIVERSITY) to prepare culture solution. Also, the methane fermentation digestive solution from cattle manure was centrifuged and diluted 20-fold, 50-fold and 100-fold with distilled water to prepare 20-fold diluted digestive solution, 50-fold diluted digestive solution and 100-fold diluted digestive solution, respectively. Each of the prepared culture solutions (4 mL) was collected and the fluorescence intensity thereof was measured for 12 days with a fluorescence spectrophotometer (FP-6600, JASCO Corporation) with an excitation wavelength of 436 nm and a fluorescence wavelength of 684 nm. In addition, the light transmittance was measured for each diluted digestive solution. In the measurement, a light wavelength of 784 µm was used, and the light transmittance in the state that distilled water is placed in a 1 cm quartz cell was defined as 100%.

The changes over time in the fluorescence intensity for each culture solution are show FIG. 2. In the 50-fold and 100-fold diluted digestive solutions, a tendency of increasing the fluorescence intensity was observed as with the CSi medium. In the 20-fold diluted digestive solution, the increase in fluorescence intensity was delayed longer than other digestive solutions and the CSi medium, but the increase in fluorescence intensity was similar.

The light transmittances for each diluted digestive solution are shown in Table 1.

TABLE 1

| | Light transmittance (%) |
|---|---|
| 20-fold diluted digestive solution | 4.7 |
| 50-fold diluted digestive solution | 34.9 |
| 100-fold diluted digestive solution | 55.9 |

The result suggested that the culture of a microalga could be performed by the use of digestive liquids whose light transmittance is 34.9% or more. In addition, it was thought that the 50-fold diluted digestive solution with the highest fluorescence intensity was optimal for the culture of microalga.

### Example 2: Study of Factors That Inhibit Light Transmission

The digestive solution was filtrated by a membrane filter with a pore size of 1 µM or 0.45 µM and the light transmittances for undiluted digestive solution and the filtrates filtered by each filter were measured by a spectrophotometer (U-1800, Hitachi High-Tech Science Corporation). In addition, 1 g or 0.25 g of granular activated carbon was added into 50 mL of the digestive solution and the mixture was shaken at 200 rpm for at least 0.5 h. The reaction solution was then filtrated in a similar method to the above filtration method and the light transmittances for each filtrate were measured. In the measurement, a light wavelength of 684 µm was used, and the light transmittance in the state that distilled water is placed in a 1 cm quartz cell was defined as 100%.

The light transmittances for the undiluted digestive liquid and the filtrates are shown in Table 2.

TABLE 2

| Filter pore size | | 1 µm | 0.45 µm |
|---|---|---|---|
| | Undiluted digestive solution | Filtrate | Filtrate |
| undiluted solution | 0 | 0 | 40.6 |
| Addition of granular activated carbon (1 g/50 mL) | - | 0 | 80.0 |
| Addition of granular activated carbon (0.25 g/50 mL) | - | 0 | 78.4 |

As shown in the above result, the light transmittance for the undiluted digestive solution was zero, and the light transmittance for the filtrate from the 1 µm filter was not changed regardless of the amount of activated carbon. On the other hand, the light transmittance for the filtrate from the 0.45 µm filter was improved by the addition of activated carbon as compared to the undiluted digestive solution. These results suggested that coloring components (0.45 µm or less) were adsorbed on the activated carbon. In addition, the light transmittance for the filtrate from the 1 µm filter was not improved. The result showed that the light transmittance was not improved as long as particles with a size of 1 µm or less were contained in digestive solution even if coloring components were removed.

The results showed that in the digestive solutions, turbidity components with larger particle size than coloring components inhibited the light transmission, and thus it was necessary to separate the turbidity components and nutrient salts in the culture of alga,. Also, it was shown that it was helpful to use the filler with a pore size of 0.45 µM in the separation of the turbidity components and the nutrient salts.

### Example 3: Effect of Mixed Gas (CO₂ Gas) Addition in Culture of Indigenous Microalga

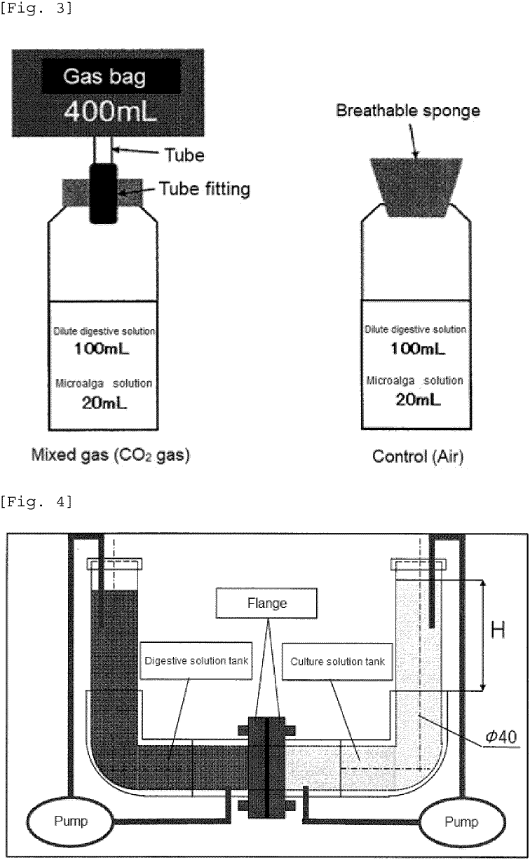

The digestive solution obtained from biomass plant (BGP) from cattle manure was centrifuged and diluted 50-fold with distilled water so that the light transmittance with a wavelength of 684 nm was 28%, and then KH₂PO₄ was added thereto as phosphorus source at 60 mg/L to prepare dilute digestive solution. Indigenous microalga solution was prepared by pre-culturing an indigenous microalga in environmental water collected from the bottom of Ono pond located on the campus of HOKKAIDO UNIVERSITY with the dilute digestive solution for about 10 days and the prepared solution was used. The dilute digestive solution (100 mL) and the indigenous microalga solution (20 mL) were added in a vial with butyl rubber aluminum seal stopper (Volume: 228 mL), and the effect of culturing an indigenous microalga by the addition of CO₂ was evaluated by a vial with an aluminum gas bag filled with 400 mL of gas in which CO₂ gas and air were mixed and the concentration of CO₂ gas was adjusted to about 10% (GL Sciences) connected with a tube and a tube fitting (left side of FIG. 3). As a control, the effect of culturing an indigenous microalga by air addition was evaluated by a vial stoppered with breathable silicone filled with the dilute digestive solution (100 mL) and the indigenous microalga solution (20 mL) (right side of FIG. 3). Each vial was cultured under the culture condition in Table 3.

TABLE 3

Culture condition of indigenous microalga

| | Air culture | CO₂ gas culture |
|---|---|---|
| Contact gas with culture solution | Air | Mixed gas (CO₂ gas concentration: 12.32%) |
| Concentration of phosphorus source in culture solution [mol/m³] (KH₂PO₄ addition) | 0.232 | |
| Photon flux density [µmol/m²/s] (on the surface of culture solution) | 207 | |
| Depth of culture solution [cm] | 6.35 | |
| Surface Area [cm²] | 18.9 | |
| Culture Temperature [°C] | 23 | |
| Light transmittance of medium [%] | 28.1 | |
| Initial indigenous microalga concentration [mg/L] | 0.07 | |
| Initial NH₄+ concentration [mg-NH₄/L] | 45.6 | |

The results of air culture and CO₂ gas culture are shown in Table 4.

TABLE 4

| | Air culture | CO₂ gas culture |
|---|---|---|
| Indigenous microalga concentration after culture [mg/L] | 176 | 250 |
| Mean growth rate [g/m³/d] | 49 | 73 |
| Consumed NH₄ amount per grown alga [g/g] | 0.074 | 0.062 |

As shown in Table 4, the concentration of indigenous microalga in the CO₂ gas culture by mixed gas was higher as compared to the concentration of indigenous microalga in the air culture. This would be because the amount of CO₂ gas supplied from mixed gas is larger than that supplied from air.

Hence, it was suggested that the culture of alga was activated by the supply of CO₂ to the alga.

### Example 4: Separation Test of Turbidity Components and Nutrient Salts in Digestive Solution

The light transmittance for culture solution as well as the amounts of ammonium ion (NH₄+) and potassium ion (K⁺) in digestive solution and culture solution were measured by an experimental apparatus shown in FIG. 4 to confirm whether turbidity components and nutrient salts are separated. Specifically, the digestive solution tank and the culture tank was separated by 0.45 µm micro filtration (MF) membrane in a clear pipe with a diameter of 40 mm made from polyvinyl chloride connected with a flange with a packing and the filter between the separated flanges, 600 mL each of digestive solution and distilled water was added to each of the tanks, respectively, and the volume of each solution was kept at the same level. In order to stir the inside of each tank, each solution was circulated from the bottom to the top of each tank at 400 mL/min by each pump. The solution position H was measured over time, 5 mL each of the solution from both tanks was collected, and the light transmittance for the culture solution was measured using a fluorescence spectrophotometer of a fluorescence wavelength of 684 nm (n=2). Also, the concentrations of ammonium and potassium ions in both solutions were measured (n=2). The test period is 7 days.

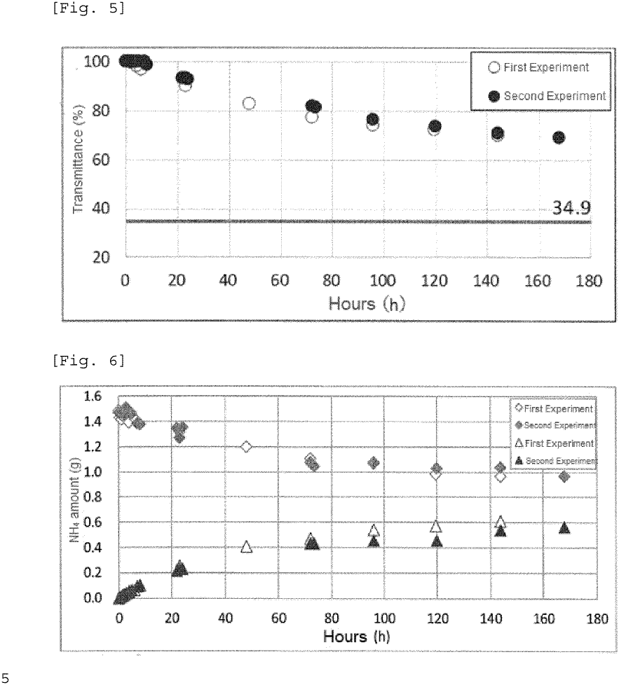

The changes over time in the light transmittances for the culture solution are shown in FIG. 5. The light transmittances were decreased, but the change was gradually small. This would be because the coloring components with a particle size of less than 0.45 µm penetrate the membrane and results in a smaller difference in concentration between the two tanks, and thus the movement rate of the components is smaller.

The light transmittance for the culture solution was higher than that of the 50-fold diluted digestive solution optimal for culturing a microalga shown in Example 1 (34.9%). Hence, the results showed that the movement of turbidity components which inhibits the culture of alga was minimized.

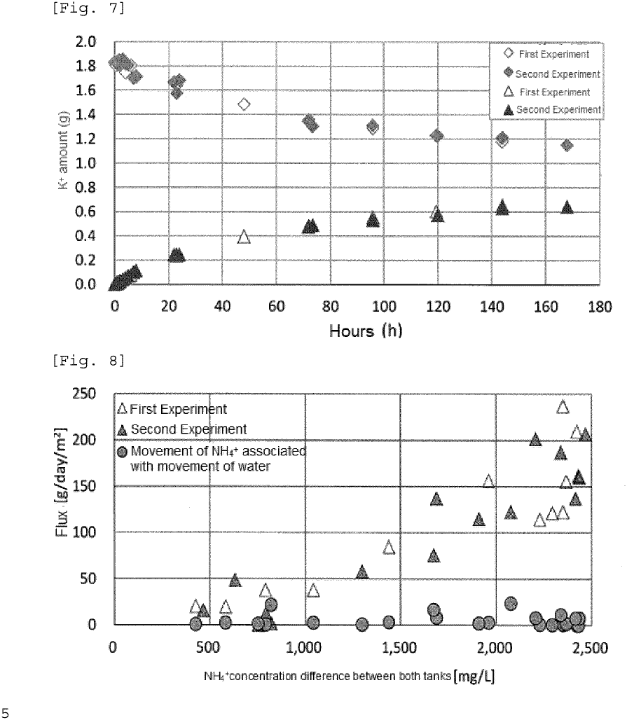

The changes over time in the concentrations of ammonium ion and potassium ion in the digestive solution tank and the culture solution tank are shown in FIGS. 6 and 7, respectively. The concentrations of both NH₄+ and K⁺ ions in the culture solution tank were increased over time and the increase in concentration was gradually small. On the other hand, the concentrations thereof in the digestive solution tank were decreased. The results showed that nutrient salts were supplied from the digestive solution tank to the culture solution tank.

The amount of nutrient salts moved from the digestive solution tank to the culture tank per unit time and unit area (separation flux) and the amount of NH₄+ associated with the movement of water from the culture tank to the digestive solution tank (movement flux) are shown in FIG. 8. The separation flux was calculated from the difference in concentration of the nutrient salts between both tanks and the change in the concentration of the nutrient salts in the culture tank to observe the movement of the nutrient salts produced by the concentration difference. The surface of solution in the tank tended to be higher on the culture tank side over time, and the surface was observed to reach 3 to 4 cm. This would be because the concentration of solutes on the digestive solution side is higher than that of the culture solution side, and thus the osmotic pressure difference was produced between both tanks and water moved to the digestive solution side.

As shown in FIG. 8, the movement flux was sufficiently smaller than the separation flux to the culture tank side. Hence, it was suggested that the separation flux to the culture tank side was dominantly produced by the diffusion movement driven by the difference in concentration of NH₄+ between the tanks. Also, a linear relationship was observed between the difference in concentration of NH₄+ and the separation flux, although there was some variation. As a result, it was found that the separation depended on the difference in concentration of NH₄+. According to the linear approximation method, the slope was 0.087 m/d and the correlation coefficient was 0.908.

### Example 5: Study of Large-Scale Culture of Microalga

In the alga culture system shown in FIG. 1, the growth rate of microalga was predicted using digestive solution obtained from a scale of 100 dairy cows with the unit volume as one unit, the volumes of each tank as 1 m³ and the parameters for the generation of digestive solution shown in Table 5. The amounts of feces and urine and the moisture content of digestive solution used the parameters described in New Energy Foundation: Biomass Engineering Handbook, p.240 (2008), Ohm-sha and Heinz Schulz, Barbara Eder: Biogas-Praxis p.135 (2002), respectively. Also, the parameter for the NH₄ concentration in the digestive solution used the observed value from an ion chromatography analyzer (DIONEX DX - 120, Thermo Fisher Scientific K.K.).

TABLE 5

| | |
|---|---|
| Amount of feces and urine generated | 58.9 kg/day/head |
| Ratio of digestive solution generated | 1 kg-digestive solution/kg-feces and urine |
| Moisture content of digestive solution | 93.24 % |
| Density of digestive solution | 1000 kg/m³ |
| NH₄ concentration of digestive solution | 2500 g/m³ |

If a biogas plant (fermenter; BGP) is used in a scale of 100 dairy cows, it is predicted that digestive solution is generated in an amount of 5.5 m³ per day and that the amount of water flowed into digestive solution tank (Q_{d}) when 1% of the generated digestive solution is used is 0.055 m³/d. When the average rates of the microalga grown by the air culture and the CO₂ gas culture and the consumed NH₄ amount per microalga shown in Table 4 were calculated according to the following formula:

$${C}_{s d} = {C}_{s d}^{i n} - \frac{{r}_{x} {Y}_{x s} {V}_{c}}{{Q}_{d}} \qquad \text{(5)}$$

the concentration of NH₄ in the digestive solution tank in the steady state was 2434 or 2418 g/m³. It showed that high concentration of NH₄ was kept. Hence, it is predicted that the higher utilization of digestive solution per unit results in a larger Q_{d} value, and thus the nutrient salts can be kept at higher concentration.

In addition, when the concentrations of each microalga after the air culture and the CO₂ gas culture are calculated according to the following formula:

$${C}_{x} = \frac{{r}_{x} {V}_{c}}{{Q}_{c}} \qquad \text{(7)}$$

the flowed amounts are 0.28 and 0.29, respectively. It showed that the volume of the culture solution did not exceed 1 m³. In batch culture test, the growth of microalga was observed in a NH₄+ amount of 1.55 g per the initial amount of microalga. As a result, it is expected that the culture is not in rate-limiting state and can be done in steady state when the NH₄+ amount per the initial microalga is maintained.

In view of the calculated results, the concentration of NH₄+ for the concentrations of microalga shown in Table 4 in constant culture for achieving the same amount of NH₄+ per microalga as in the initial stage is 272 or 387 g/m³. In order to achieve the steady concentration in the culture tank, the area of membrane calculated according to the following formula is 0.0193 or 0.0256 m².

$${C}_{s c} = {C}_{z d}^{i n} - \frac{{r}_{x} {Y}_{x c} {V}_{c}}{{Q}_{d}} - \frac{{r}_{x} {Y}_{x c} {V}_{c}}{k {A}_{f}} \qquad \text{(6)}$$

The movement rate coefficient of membrane is defined as 0.087 m/s (slope of the appropriate straight line shown in FIG. 5) .

According to the above results, the concentration of microalga in the culture tank for growing the microalga is kept and the concentration difference between both tanks is constantly kept, and thus a culture rate of microalga of 49 or 73 g/m³/d can be achieved. In such case, a membrane area of 0.0193 or 0.0256 m² or more is required per unit of 1 m³ and the separation rate of NH₄⁺ is 188 or 177 g/m²/d. The separation rate is lower as the membrane areas increases.

The alga culture system and the method of culturing an alga of the present invention can produce the supply rate of nutrient salts while achieving a usual culture rate of alga. They enable continuous culture of the alga, and thus can achieve the culture and extraction of a large amount of alga.

### Example 6: Effect of Nutrient Salts on Culture of Microalga

In this study, the concentrations of nutrient salts and microalga were measured and analyzed using an apparatus equipped with a digestive solution tank, micro filtration membrane with a pore size of 0.45 µm and 10 L of a basin (culture tank) to study the effect of the nutrient salts on the culture of microalga.

Specifically, the concentration of microalga in the culture tank as well as the concentrations of NH₄⁺ and PO₄³⁻ in digestive solution and culture solution were measured according to the following procedures. In this study, an indigenous microalga was used as the microalga.

### Preparation of Culture Solution (Medium)

A digestive tank was filled with 113 mL of the methane fermentation digestive solution from cattle manure, 5000 mL of distilled water was added into a basin (culture tank), and then an apparatus comprising the digestive solution and a membrane was placed within the culture tank. The solution in the culture tank at a temperature of 26° C. was then stirred using a stirrer (NZ-1200, TOKYO RIKAKIKAI CO., Ltd.) at 190 rpm for 5 days to prepare a culture solution.

The entire culture tank was placed on an electronic scale to measure the evaporated amount of distilled water, and distilled water was randomly added to bring the solution volume to 5000 mL.

### Measurement of the Concentration of Microalga in the Culture tank and the Ions (NH₄+ and PO₄³-) Amounts in Digestive Solution and Culture Solution

The pre-cultured microalga was inoculated into the culture solution prepared in the above (1). The inoculated culture solution was cultured under the culture condition shown in Table 6 using a stirrer (NZ-1200, TOKYO RIKAKIKAI CO., Ltd.) at 250 to 300 rpm for 28 days. The concentration of microalga in the culture tank and the PO₄³⁻ amounts in digestive solution and culture solution were measured by sampling 1 mL of the digestive solution from the digestive solution tank and 10 mL of the culture solution from the culture tank every 24 hours after the culture of culture solution. In addition, the NH₄⁺ amounts in the digestive solution and culture solution were measured to study whether or not microalga was cultured using the nutrient salts in the digestive solution. The concentration of microalga was calculated from the weight measured after the microalga in the culture solution were extracted through the micro filtration membrane with a pore size of 0.45 µm and dried at 105° C. for 24 hours, and the PO₄³⁻ and NH₄⁺ amounts were measured by the ion chromatography (DIONEX DX-120, Thermo Fisher Scientific K.K) or the ion chromatography (IC-2010, TOKYO KAKEN CO., Ltd.).

Distilled water was added on Days 3, 6, 10, 12, 14, 17, 21 and 25, the digestive solution in the device was replaced on Days 8 and 21, and KH₃PO₄ was added on Day 14.

TABLE 6

Culture condition

| | |
|---|---|
| Volume of culture solution [mL] | 3700-5000 |
| Depth of culture solution [mm] | 55-75 |
| Light condition [µmol/m²/s] | 200-240 (Fluorescent lamp 24-hour light) |
| Culture temperature [°C] | 26 |
| Nutrient salt (KH₂PO₄) addition [g] | 0.715 |
| Initial microalga concentration [g/L] | 0.06 |
| Initial K⁺ amount [mg-K⁺/L] | 41 |
| Initial PO₄³⁻ amount [mg-PO₄³⁻/L] | 100 (1.05 mol/m³) |

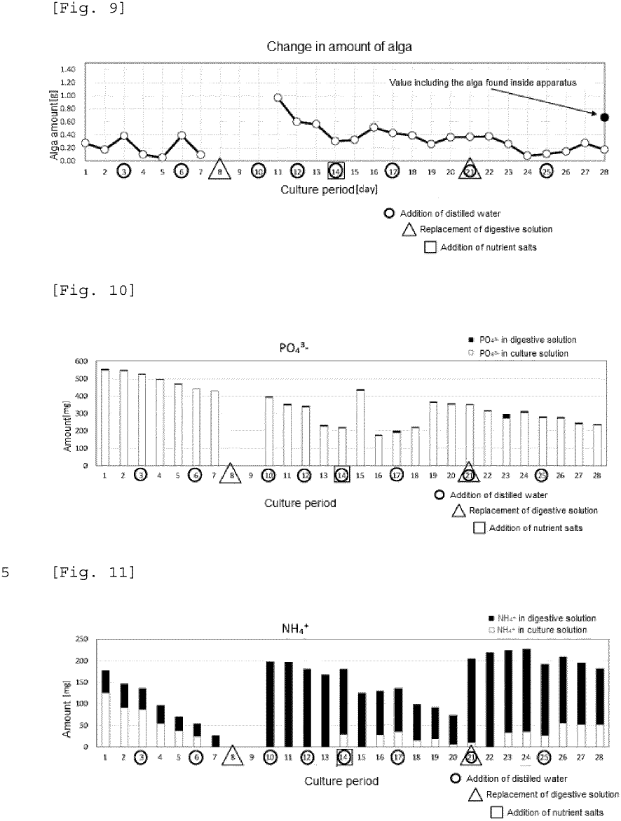

The amounts of microalga in the culture tank from Day 1 to Day 28 are shown in FIG. 9. The amounts of microalga on Day 8 to Day 10 were not measured.

The growth of microalga was not observed until around Day 9 after the inoculation of microalga, but the culture solution turned green on around Day 10 and the growth of microalga could be visually observed. On Day 12, 1L of the culture solution (microalga amount: 0.16 g) was collected. Thereafter, the amount of microalga gradually decreased, and it was visually observed that the color of the culture solution became lighter. When the membrane separator was disassembled after measuring the amount of microalga on Day 28, microalga was growing in the gaps between the flanges of the apparatus. The microalga found inside the apparatus were dropped into the culture solution with a brush, and the amount of microalga was 0.5 g.

The PO₄³⁻ amounts in digestive solution and culture solution from Day 1 to Day 20 are shown in Table 7 and FIG. 10. The PO₄³⁻ amounts on Day 8 and Day 9 were not measured.

TABLE 7

| Culture period (day) | PO₄³⁻ in digestive solution (mg) | PO₄³⁻ in culture solution (mg) |
|---|---|---|
| 1 | 5.79 | 547.63 |
| 2 | 5.41 | 544.24 |
| 3 | 2.59 | 523.93 |
| 4 | 2.73 | 494.33 |
| 5 | 3.20 | 464.58 |
| 6 | - | 440.74 |
| 7 | - | 429.69 |
| 10 | 5.10 | 389.06 |
| 11 | 7.55 | 344.55 |
| 12 | 5.00 | 333.97 |
| 13 | 5.31 | 224.83 |
| 14 | 5.42 | 212.34 |
| 15 | 5.60 | 431.02 |
| 16 | 5.59 | 170.10 |
| 17 | 5.62 | 190.04 |
| 18 | 5.63 | 214.98 |
| 19 | 5.80 | 360.54 |
| 20 | 5.91 | 350.47 |
| 21 | 4.36 | 347.51 |
| 22 | 5.24 | 311.86 |
| 23 | 24.20 | 269.46 |
| 24 | 7.36 | 304.84 |
| 25 | 5.07 | 271.45 |
| 26 | 5.25 | 269.75 |
| 27 | 5.30 | 238.57 |
| 28 | 5.46 | 231.82 |

As shown in Table 7, the digestive solution did not almost contain PO₄³⁻. Hence, it was shown that PO₄³⁻ in the culture solution was derived from the added KH₃PO₄, and PO₄³⁻ was not almost moved between the digestive solution and the culture solution. In addition, PO₄³⁻ was consumed at an almost constant rate in the culture solution. Hence, it was shown that the microalga could be cultured by the use of PO₄^{3-.}

In addition, the growth rate of microalga was calculated at regular intervals (Day 1 to Day 6, Day 10 to Day 14, Day 15 to Day 19 and Day 19 to Day 28). The calculated growth rate of microalga was 78 to 234 g/m³/d, and it was higher than the rates in air culture and CO₂ gas culture. The growth rate of microalga in each period was calculated according to (PO₄³⁻ amount in culture solution on the first day (mg) - PO₄³⁻ amount in culture solution on the last day (mg)) x amount of phosphate consumed by microalga (0.044 mg)/ Volume of solution (L)/ Days (day). For example, the growth rate of microalga from Day 1 to Day 6 is (PO₄³⁻ amount in culture solution on Day 1 (547.63 mg) - PO₄³⁻ amount in culture solution on Day 6 (440.74 mg)) x amount of phosphate consumed by microalga (0.044 mg)/ Volume of solution (3.8648 L)/ Days (5 day) = 125.71 mg/L/day (g/m³/day) .

The results suggested that the culture of an alga was activated by supplying high concentration of phosphate ions into culture solution.

The amounts of NH₄⁺ in digestive solution and culture solution from Day 1 to Day 28 are shown in Table 8 and FIG. 11. The NH₄⁺ amounts on Day 8 and Day 9 were not measured.

TABLE 8

| Culture period (day) | NH₄⁺ in digestive solution (mg) | NH₄⁺ in culture solution (mg) |
|---|---|---|
| 1 | 51.21 | 125.58 |
| 2 | 55.24 | 90.40 |
| 3 | 48.13 | 86.37 |
| 4 | 41.38 | 54.33 |
| 5 | 32.68 | 37.09 |
| 6 | 30.01 | 23.89 |
| 7 | 26.11 | ND |
| 10 | 197.80 | ND |
| 11 | 195.90 | ND |
| 12 | 180.25 | ND |
| 13 | 166.85 | ND |
| 14 | 150.78 | 29.2 |
| 15 | 123.98 | ND |
| 16 | 102.04 | 28.05 |
| 17 | 100.22 | 34.78 |
| 18 | 82.62 | 15.02 |
| 19 | 72.09 | 18.33 |
| 20 | 66.62 | 5.73 |
| 21 | 194.77 | 9.76 |
| 22 | 218.23 | 0.11 |
| 23 | 192.02 | 32.10 |
| 24 | 192.31 | 35.08 |
| 25 | 164.99 | 26.41 |
| 26 | 153.37 | 55.82 |
| 27 | 143.02 | 51.58 |
| 28 | 129.09 | 51.94 |

The decrease in the NH₄⁺ amounts in both digestive solution and culture solution was observed up to Day 7 after the inoculation of microalga. As a result, it was suggested that NH₄⁺ was transferred from digestive solution through the membrane to culture solution and that NH₄⁺ was consumed by microalga in culture solution.

After the replacement of digestive solution on Day 8, the total amount of NH₄⁺ from Day 10 to Day 13 was reduced, but NH₄⁺ was not observed in the culture solution. This would be because the amount of NH₄⁺ consumed by the grown microalga exceeded the supplied NH₄⁺ amount. On the other hand, NH₄⁺ could be observed in the culture solution after Day 13. This would be because the consumption of NH₄⁺ by microalga was reduced by the reduction in the growth rate of microalga in the culture solution and the supplied NH₄⁺ amount exceeded the consumed NH₄⁺ amount.

### INDUSTRIAL APPLICABILITY

The present invention can supply the nutrient salts from digestive solution to culture solution at the supply rate and in the required amount suitable for the culture of an alga. Also, the present invention can supply the nutrient salts without the pre-treatment processes such as the removal of turbidity components, and thus can achieve the culture and extraction of an alga at low cost.

In addition, the present invention can culture a large amount of an alga, and thus it is expected to enable the practical realization of the production of biofuels and bioenergy on a commercial scale.

1. A method of culturing an alga using a reaction tank equipped with a digestive solution tank comprising digestive solution containing high concentration of nutrient salts, a membrane with a pore size of 0.45 µm or less and a culture tank comprising culture solution and an alga, which comprises supplying the nutrient salts contained in the digestive solution through the membrane into the culture solution by the diffusion driven by the difference in concentration of the nutrient salts between the digestive solution tank and the culture tank.

2. The method according to claim 1, wherein the supply rate of the nutrient salts is 177 to 188 g-N/m² /d.

3. The method according to claim 1, wherein the membrane has an area of 0.0193 m² or more.

4. The method according to claim 1, wherein the culture rate of the alga is 49 to 234 g/m³ /d.

5. The method according to claim 1, which further comprises circulating each solution in the digestive solution tank and the culture tank using each pump further equipped in each tank and keeping the volumes of the digestive solution and the culture solution at the same level.

6. The method according to claim 1, which further comprises supplying CO₂ into the culture tank.

7. The method according to claim 1, which further comprises supplying phosphate ion (PO₄³⁻) into the culture tank.

8. The method according to claim 1, wherein the digestive solution is methane fermentation digestive solution.

9. The method according to claim 1, wherein the culture solution is tap water without chlorine, groundwater, or river or lake water.

10. The method according to claim 1, wherein the alga is a microalga.

11. The method according to claim 1, wherein the membrane is micro filtration membrane (MF membrane).

12. The method according to claim 1, wherein the nutrient salts comprises one or more salts consisting of ammonia nitrogen, nitrate nitrogen, phosphate phosphorus, orthosilicic acid, potassium, calcium, magnesium and sulfur.

13. An alga culture system comprising a digestive solution tank, a membrane with a pore size of 0.45 µm or less and a culture tank, wherein the digestive solution tank comprises digestive solution containing high concentration of nutrient salts, the culture tank comprises culture solution and an alga, and the membrane is set as a partition between the digestive solution tank and the culture tank.

14. The alga culture system according to claim 13, wherein the system maintains the difference in concentration of nutrient salts between the digestive solution tank and the culture tank produced by the consumption of the nutrient salts by the alga in the culture tank and supplies the nutrient salts contained in digestive solution into culture solution by the diffusion driven by the difference in concentration of nutrient salts.

15. The alga culture system according to claim 13 which is arranged in the order of the digestive liquid tank, the membrane and the culture tank in a horizontal direction.

16. The alga culture system according to claim 13 which is arranged in the order of the digestive liquid tank, the membrane and the culture tank in a vertical direction.

17. A method of supplying nutrient salts using a reaction tank equipped with a digestive solution tank comprising digestive solution containing high concentration of nutrient salts, a membrane with a pore size of 0.45 µm or less and a culture tank comprising culture solution and an alga, wherein comprises maintaining the difference in concentration of nutrient salts between the digestive solution tank and the culture tank produced by the consumption of the nutrient salts by the alga in the culture tank and supplying the nutrient salts contained in digestive solution into culture solution by the diffusion driven by the difference in concentration of nutrient salts.
